(19) United States
(12) Patent Application Publication
(10) Pub. No.: US 2025/0238580 A1
(43) Pub. Date: Jul. 24, 2025

(54) Systems and Methods for Augmenting Formulation Design

(71) Applicant: Kenvue Brands LLC, Summit, NJ (US)

(72) Inventor: Seng Khoon TEH, Ascent```` (SG)

(21) Appl. No.: 19/014,696

(22) Filed: Jan. 9, 2025

Related U.S. Application Data
(60) Provisional application No. 63/622,646, filed on Jan. 19, 2024.

(51) Int. Cl. G06F 30/27

(52) U.S. Cl. CPC G06F 30/27

(57) ABSTRACT

Systems and methods for augmenting formulation design. The method includes determining a functional ingredient category for an individual ingredient in a requested new formulation; identifying a representative molecular structure of the individual ingredient in the determined functional ingredient category; generating, by a trained machine learning (ML) model, a predicted set of desired properties of i) the requested new formulation and ii) the identified representative molecular structure of the individual ingredient in the determined functional ingredient category; and generating, based on the generated predicted set of desired properties, a recommended predicted formulation that includes the identified representative molecular structure corresponding to the set of desired properties of the requested new formulation.

### CROSS-REFERENCE TO RELATED APPLICATION

This application claims the benefit of U.S. Provisional Application No. 63/622,646 filed Jan. 19, 2024, the contents of which is incorporated herein by reference in its entirety.

### BACKGROUND

Predictive chemical formulation is an emerging, data-driven approach that can provide a formulation chemist with recommendations of novel formulations with the highest propensity to simultaneously fulfil multiple targeted properties, reducing the need for cost- and time-consuming trial-and-error experiment and accelerating the speed of consumer health formulation innovation. Current methods of predictive chemical formulation generally include the use of either functional categories or chemical characteristics as data features for supervised machine learning (ML). The functional category method essentially leverages on the formulation chemist's experience to manually labeled each ingredient with a main chemical property. Though this approach exploits the expert domain knowledge, it is prone to recall bias and is subject to experience bias from the individual formulation chemist, causing significant challenges in its implementation. Other emerging methods, including the functional category label-free approaches, e.g., those based on chemical characteristics, suffer from low Signal-to-Noise (S/N) ratios due to challenges in determining the optimal set of features in an unprecedented amount of large, high-dimensional space, minimizing their predictive efficacy.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one example, a computer-implemented method is provided. The method includes receiving a request for a new formulation having a set of desired properties; determining a functional ingredient category for an individual ingredient in the requested new formulation; identifying a representative molecular structure of the individual ingredient in the determined functional ingredient category; generating, by a trained machine learning (ML) model, a predicted set of desired properties of i) the requested new formulation and ii) the identified representative molecular structure of the individual ingredient in the determined functional ingredient category; and generating, based on the generated predicted set of desired properties, a recommended predicted formulation, the predicted formulation including the identified representative molecular structure corresponding to the set of desired properties of the requested new formulation.

In another example, a system is provided. The system includes a memory, a transceiver to receive a request for a new formulation having a set of desired properties, and a processor coupled to the memory. The processor is configured to determine a functional ingredient category for an individual ingredient in the requested new formulation; identify a representative molecular structure of the individual ingredient in the determined functional ingredient category; generate, by a trained machine learning (ML), a predicted set of desired properties of i) the requested new formulation and ii) the identified representative molecular structure of the individual ingredient in the determined functional ingredient category; and generate, based on the generated predicted set of desired properties, a recommended predicted formulation, the recommended predicted formulation including the identified representative molecular structure corresponding to the set of desired properties of the requested new formulation.

In another example, a computer-readable storage media is provided. The computer-readable storage media stores instructions that, when executed by a processor, cause the processor to control to receive a request for a new sunscreen protection factor (SPF) formulation having a set of desired properties; determine a functional ingredient category for an individual ingredient in the requested new formulation, wherein the functional ingredient category includes at least one of an SPF value, a UVA filter, or a UVB filter; identify a representative molecular structure of the individual ingredient in the determined functional ingredient category; generate, by a trained machine learning (ML), a predicted set of desired properties of i) the requested new SPF formulation and ii) the identified representative molecular structure of the individual ingredient in the determined functional ingredient category; and generate, based on the generated predicted set of desired properties, a recommended predicted SPF formulation, the recommended predicted SPF formulation including the identified representative molecular structure corresponding to the set of desired properties of the requested new SPF formulation.

### BRIEF DESCRIPTION OF THE DRAWINGS

The present description will be better understood from the following detailed description read in light of the accompanying drawings, wherein:

FIG. 1 illustrates an example system for determining an ingredient functional category and generating a recommended predicted formula;

FIG. 2 illustrates an example system for determining a functional ingredient category, generating a prediction, and storing a generated data structure;

FIGS. 3A-3B illustrate an example computer-implemented method of determining an ingredient functional category and generating a recommended predicted formula;

FIG. 4 illustrates an example computer-implemented method of determining a functional ingredient category;

FIG. 5 illustrates an example computer-implemented method of generating a recommended predicted formula;

FIGS. 6A-6B illustrate an example computer-implemented method of generating a recommended predicted formula having a requested sunscreen protection factor (SPF); and

FIG. 7 is a block diagram illustrating an example computing environment suitable for implementing one or more of the various examples disclosed herein.

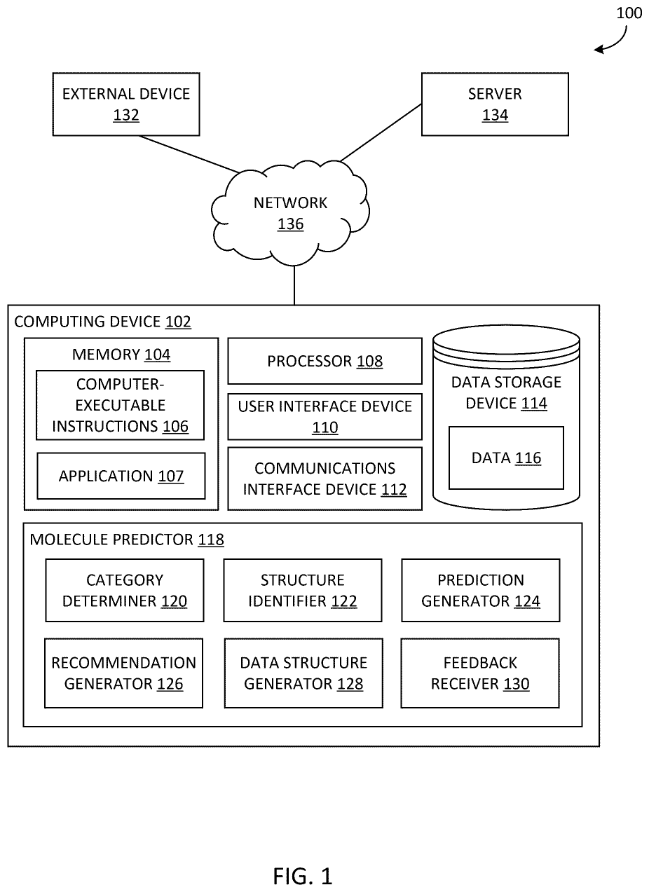

Corresponding reference characters indicate corresponding parts throughout the drawings. In FIGS. 1 to 7, the systems are illustrated as schematic drawings. The drawings may not be to scale.

### DETAILED DESCRIPTION

The various implementations and examples will be described in detail with reference to the accompanying drawings. Wherever possible, the same reference numbers will be used throughout the drawings to refer to the same or like parts. References made throughout this disclosure relating to specific examples and implementations are provided solely for illustrative purposes but, unless indicated to the contrary, are not meant to limit all examples.

As described herein, current methods of generating chemical formulations are manual, labor-intensive, time-intensive, expensive, and prone to bias and user error. While some methods of recommending a novel formulation include technical solutions such as sequential learning, the search space to be used for recommending a novel formulation is vast. Sequential learning is an emerging machine learning approach that accurately selects a material chemistry candidate with the highest likelihood to meet the initial product design specification for testing. This could potentially lead to a reduced number of experimental iterations, accelerating product design innovation cycle and product time-to-market, but current iterations that incorporate sequential learning in isolation fail to narrow the search space to a degree the technology is beneficial. Thus, there remains a need to determine systems and methods for defining and redefining a targeted search space for incorporation into a sequential learning platform.

As referenced herein, current methods of predictive chemical formulation generally include the use of either functional categories or chemical characteristics as data features for supervised machine learning (ML). The functional category method leverages on an individual formulation chemist's experience to manually label each ingredient with a main chemical property. Though this approach exploits the expert domain knowledge, it is prone to recall bias and is subject to experience bias from the individual formulation chemist, causing significant intra- and inter-rater's variability in the labeling of the functional category of ingredients. This in turn affects the data accuracy for downstream predictive chemical formulation tasks. In addition, this method fails to take into account the fact that different types of ingredients in the same functional category do not always provide exactly the same functional category effect, and that the chemical interaction effect of an ingredient with different set of ingredients may not always yield the same functional category effect.

More recent functional category label-free approaches have been put forth which involve the extraction of various chemical characteristics of ingredients for chemo-informatics-driven formulation modeling. This generally involves the matching of International Nomenclature Cosmetic Ingredient (INCI) names or Chemical Abstracts Service (CAS) numbers with evolving molecular representations such as Simplified Molecular-Input Line-Entry System (SMILES) or SELF-referencing Embedded Strings (SELFIES), followed by molecular descriptor, also referred to as chemical characteristic, transformations including RDKit or Mordred, providing hundreds to thousands of chemical characteristics for each ingredients. However, this results in an extensive number of chemical characteristics from numerous ingredients in a single formulation that can be combined in an unprecedented large number of ways. This complicates the determination of an optimal set of chemical characteristics that are typically associated with low Signal-to-Noise (S/N) ratio of molecular signals linked with the targeted property, limiting the predictive accuracy.

Often times, in limited formulation sample data, the Signal-to-Noise (S/N) ratio of molecular signals that are linked with the targeted property is very low, resulting in low predictive accuracy.

The expert domain knowledge involving the functional category labeling approach and the functional category label-free approach that involves the extraction of various chemical characteristics of ingredients can be complementary techniques, rather than competitive. Consequently, there is a need for a new method that can systematically combine both approaches. In specific, there is a need for improved systems and methods of generating accurate data features with different ingredients for use by predictive models, and for improved algorithms of identifying a particular set of chemical characteristics of the molecular structures belonging to the various functional categories that can augment the accuracy of the predictive models.

This is seen in various specific use cases. For example, Sunscreen Protection Factor (SPF) models typically leverage a physics-based simulation model to simulate different types of photo-absorption interaction effects on biological tissues to determine a SPF value/factor. As this is based on a theoretical model and not based on empirical data, the estimated SPF value/factor often significantly deviates from experimental result. Current models constructed from empirical data are mainly based on the concentration of the UV filters, i.e., a functional category, of the ingredients. However, this method fails to take into account that different types of ingredients in the same functional category, i.e., UV filters, do not always provide exactly the same optical absorbance effect, and fails to account for the chemical interaction effect that may not always yield additive optical absorbance effect. In other words, while two ingredients may include the same UVA or UVB filters, they may not yield the same optical absorbance effect and interact with other ingredients in the same way.

Various examples of the present disclosure recognize and take into account these challenges and provide systems and methods for generating a chemical formulation that determine a functional ingredient category, generate a set of desired properties for the formulation based on the determined functional ingredient category, and generate a recommendation of a predicted formulation that has the generated set of desired properties. In particular, the systems and methods of the present disclosure provide an approach based on empirical data that first learns the relationship of the ingredient functional category with a desired set of properties, such as in vivo SPF, and leverages on this latent macromolecular relationship to further construct a prediction model, such as an SPF prediction model, at a deeper, microchemical/molecular level. This enables different characteristics, such as the optical absorption and chemical reaction effect of the specific ingredient within the same functional category, to be decoupled and each analyzed separately, leading to a more robust novel formulation recommendation.

The systems and methods of the present disclosure operate in an unconventional manner by implementing multiple predictive models that operate in conjunction to identify a functional ingredient category of a requested new formulation, generating a set of desired properties based on the identified functional ingredient category, determining synergistic functions of potential ingredients in a potential formulation corresponding to the identified functional ingredient category, generating a recommended formulation based on the identified functional ingredient category and determined synergistic functions, and executing each model to perform additional learning throughout in order to continuously improve the recommendation and determination processes. The implementation of separate models at each stage that operate independently yet symbiotically operate in an unconventional manner and provide an improved method of understanding a request for a new formulation and providing a tailored response based on continuous learning. As noted above, this process additionally enables different characteristics of a group of ingredients to be decoupled and analyzed sequentially, leading to a more robust novel formulation recommendation.

The systems and methods of the present disclosure provide a technical solution to the inherently technical problem of creating and storing data structures that include desired properties of a formulation along with associated functional ingredient categories and associated formulation predictions. Current storage methods lack quarriable data structures that store one or more aspects of this data, including but not limited to initially desired properties of a formulation, extracted ingredient information, resulting determined functional ingredient category information, and recommended predicted formulation information along with standardized ingredient information of the same, such as International Nomenclature Cosmetic Ingredient (INCI) names. The systems and methods of the present disclosure provide a technical solution to this inherently technical problem by providing such a data structure along with generated machine learning (ML) or artificial intelligence (AI) models that are trained and retrained using the provided data structures to continuously provide improved and updated recommendations.

In one example, the present disclosure provides methods and systems for determining an ingredient functional category of an Information Handling System (IHS). A database is used to store the knowledge base data primarily linking ingredients with functional categories from different formulation chemists and from publicly available datasets. Based on a user input that includes the product type, formulation index, the ingredients of interest using International Nomenclature Cosmetic Ingredient (INCI) names, and so forth, the single or multiple functional categories associated with each ingredient of interest are retrieved and sorted in accordance with some algorithms. The algorithms are machine learning (ML) or artificial intelligence (AI) models that generate the ranking in accordance with the number of times different users previously selected the functional category associated with the ingredient, a probability index representing the likelihood of a formulator to select the specific functional category based on prior database information that the ingredient is associated with a list of functional categories, and so forth. The most relevant functional category is visually presented in a way that the user can easily select. Based on a determination, such as by a formulation chemist or another ML or AI model, of a functional category for an ingredient for a certain formulation of a product type, this selection further updates the database. This creates a database to iteratively learn and recommend the most relevant functional category for an ingredient that capitalizes on institutional knowledge.

In some examples, determining the ingredient functional category includes extracting open-source or publicly available data and/or company formulator information from the database, defining an ingredient to use in the formulation, returning values for each query input in order of counts, quantity, or probability, and generating the recommendation. In some examples, some ingredients are flagged so as to not be recommended regardless of their status of being returned, for example due to an ingredient having a low sustainability score, a high-risk score such as due to low availability, and so forth.

FIG. 1 illustrates an example system for determining an ingredient functional category and generating a recommended predicted formula. The system 100 illustrated in FIG. 1 is provided for illustration only. Other examples of the system 100 can be used without departing from the scope of the present disclosure. In some examples, the system 100 augments formulation design according to one or more examples provided herein.

The system 100 includes a computing device 102, an external device 132, a server 134, and a network 136. The computing device 102 represents any device executing computer-executable instructions 106 (e.g., as application programs, operating system functionality, or both) to implement the operations and functionality associated with the computing device 102. The computing device 102 in some examples includes a mobile computing device or any other portable device. A mobile computing device includes, for example but without limitation, a mobile telephone, laptop, tablet, computing pad, netbook, gaming device, and/or portable media player. The computing device 102 can also include less-portable devices such as servers, desktop personal computers, kiosks, or tabletop devices. Additionally, the computing device 102 can represent a group of processing units or other computing devices.

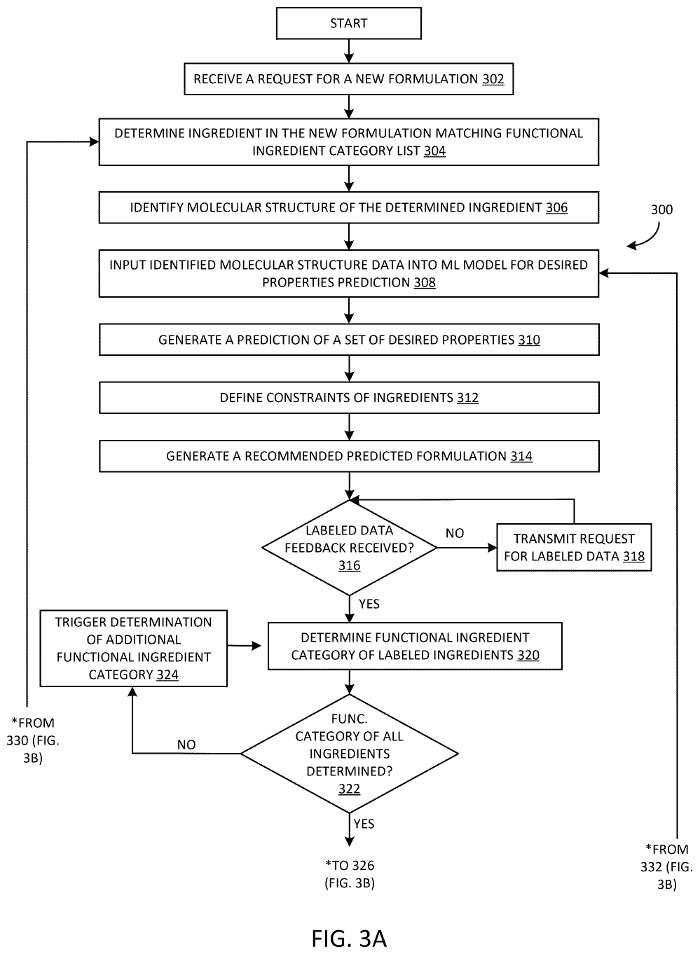

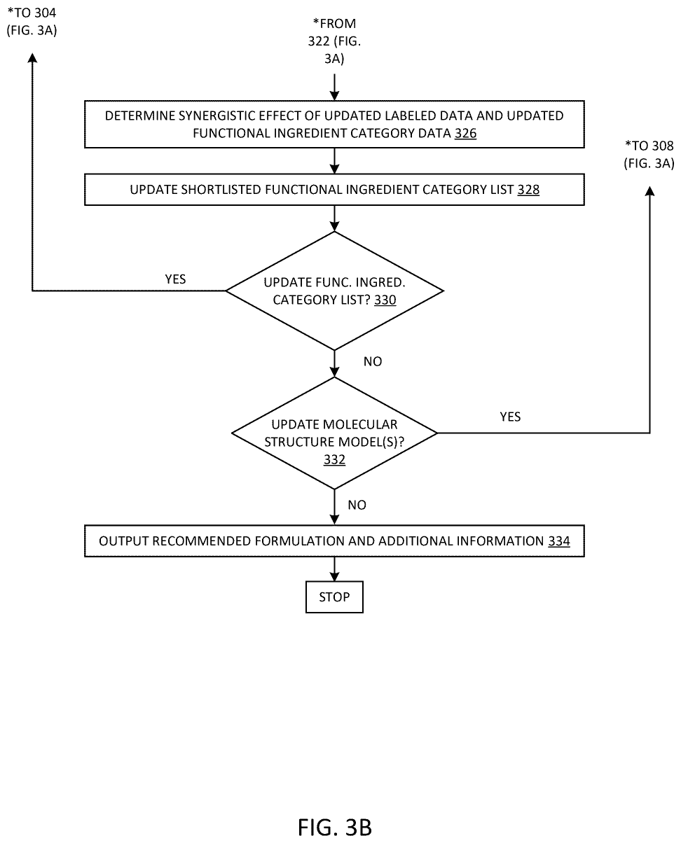

In some examples, the computing device 102 includes at least one processor 108, a memory 104 that includes the computer-executable instructions 106, and a user interface device 110. The processor 108 includes any quantity of processing units and is programmed to execute the computer-executable instructions 106. The computer-executable instructions 106 are performed by the processor 108, performed by multiple processors within the computing device 102, or performed by a processor external to the computing device 102. In some examples, the processor 108 is programmed to execute computer-executable instructions 106 such as those illustrated in the figures described herein, such as FIGS. 3A-6B. In various examples, the processor 108 is configured to execute computer-executable instructions of the molecule predictor 118 as described herein.

The memory 104 includes any quantity of media associated with or accessible by the computing device 102. In some examples, the memory 104 is internal to the computing device 102. In other examples, the memory 104 is external to the computing device 102 or both internal and external to the computing device 102. For example, the memory 104 can include both a memory component internal to the computing device 102 and a memory component external to the computing device 102, such as the server 136. The memory 104 stores data, such as one or more applications 107. The applications 107, when executed by the processor 108, operate to perform various functions on the computing device 102. The applications 107 can communicate with counterpart applications or services, such as web services accessible via the network 138. In an example, the applications 107 represent server-side services of an application executing in a cloud, such as a cloud server 136. In some examples, the application 107 is an application for predicting a molecule, or formulation including more than one molecule, as described herein. The user interface device 110 includes a graphics card for displaying data to a user and receiving data from the user. The user interface device 110 can also include computer-executable instructions, for example a driver, for operating the graphics card. Further, the user interface device 110 can include a display, for example a touch screen display or natural user interface, and/or computer-executable instructions, for example a driver, for operating the display. The user interface device 110 can also include one or more of the following to provide data to the user or receive data from the user: speakers, a sound card, a camera, a microphone, a vibration motor, one or more accelerometers, a BLUETOOTH® communication module, global positioning system (GPS) hardware, and a photoreceptive light sensor. In a non-limiting example, the user inputs commands or manipulates data by moving the computing device 102 in one or more ways.

The computing device 102 further includes a communications interface device 112. The communications interface device 112 includes a network interface card and/or computer-executable instructions, such as a driver, for operating the network interface card. Communication between the computing device 102 and other devices, such as but not limited to the external device 132, can occur using any protocol or mechanism over any wired or wireless connection.

The computing device 102 further includes a data storage device 114 for storing data 116. The data 116 includes, but is not limited to, a predicted set of desired properties of i) the requested new formulation and ii) the identified representative chemical characteristic of the individual ingredient in the determined functional ingredient category; and a generated data structure 212 as described in greater detail below that includes a determined set of INCI names, an associated set of desired properties, extracted ingredient information, determined functional ingredient category, identified representative molecular structure of the individual ingredient, and recommended predicted formulation; one or more shortlists of functional ingredient categories associated with common parameters requested for new formulations; an ingredient information library as described in greater detail below; a set of ingredients of interest using INCI names, and so forth.

The data storage device 114 may include one or more different types of data storage devices, such as, for example, one or more rotating disks drives, one or more solid state drives (SSDs), and/or any other type of data storage device. The data storage device 114 in some non-limiting examples includes a redundant array of independent disks (RAID) array. In other examples, the data storage device 114 includes a database. The data storage device 114, in this example, is included within the computing device 102, attached to the computing device 102, plugged into the computing device 102, or otherwise associated with the computing device 102. In other examples, the data storage device 114 includes a remote data storage accessed by the computing device 102 via the network 136, such as the server 134 which may be a remote data storage device, a data storage in a remote data center, a cloud storage, and so forth.

The computing device 102 further includes a molecule predictor 118. The molecule predictor 118 is an example of a specialized processor, or processing unit, implemented on the processor 108. The molecule predictor 118 predicts a molecule, or multiple molecules, in a formulation according to one or more examples described herein. The molecule predictor 118 includes a category determiner 120, a structure identifier 122, a prediction generator 124, a recommendation generator 126, a data structure generator 128, and a feedback receiver 130. Each of the category determiner 120, the structure identifier 122, the prediction generator 124, the recommendation generator 126, the data structure generator 128, and the feedback receiver 130 is an individual example of a specialized processor, or processing unit, implemented on the processor 108.

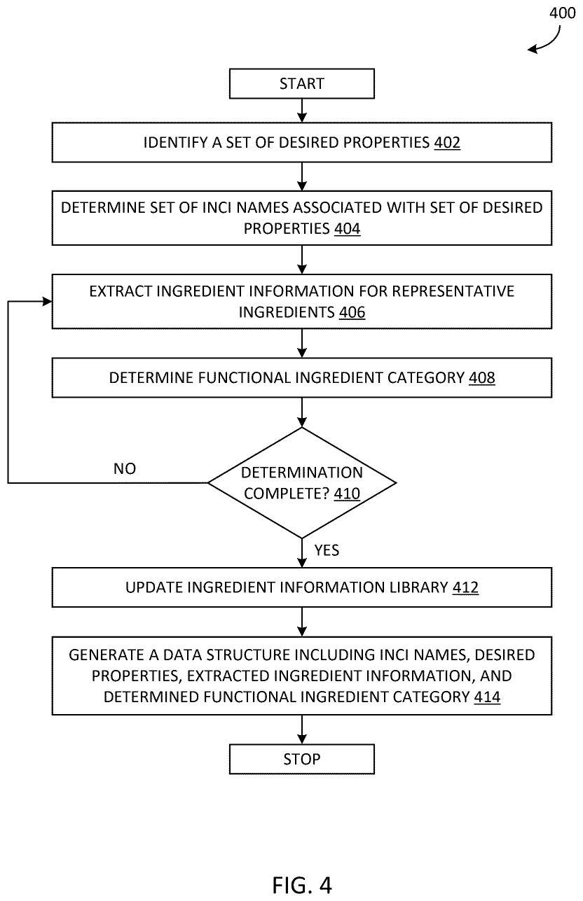

The category determiner 120 is an example of a machine learning (ML) or artificial intelligence (AI) model that determines a functional ingredient category for an individual ingredient, or multiple ingredients, in a requested new formulation. In some examples, the category determiner 120 is referred to as a first ML or AI model. In some examples, a request for a new formulation may be received either via the user interface device 110 or from an external device 132 via the communications interface device 112. The category determiner 120 determines the functional ingredient category for the ingredient, or ingredients, to be included in the recommended formulation based on the received request for the new formulation. In some examples, the category determiner 120 determines a set of nomenclature names, such as International Nomenclature Cosmetic Ingredient (INCI) names, associated with a determined set of desired properties, the set of INCI names being the individual ingredient, extracts, from a data source, ingredient information for the individual ingredient, and based on the extracted ingredient information, determines the functional ingredient category for the requested new formulation. In some examples, the category determiner 120 further evaluates test results, including but not limited to in vivo test results, in vitro test results, and so forth, and determines the functional ingredient category based on the extracted ingredient information. The process by which the category determiner 120 determines a functional ingredient category is described in greater detail below with regards to FIGS. 3A-3B and FIG. 4.

The structure identifier 122 is an example of a ML or AI model that identifies a representative molecular structure of the individual ingredient in the determined functional ingredient category. In some examples, the structure identifier 122 is referred to as a second ML or AI model. The process by which the structure identifier 122 identifies the representative molecular structure is described in greater detail below with regards to FIGS. 3A-3B and FIG. 4.

The prediction generator 124 is an example of a ML or AI model that generates a prediction of the requested new formulation. In some examples, the prediction generator 124 is referred to as a third ML or AI model. The prediction generator 124 implements a trained ML model to generate a predicted set of desired properties of the requested new formulation and of the identified representative molecular structure of the individual ingredient in the determined functional ingredient category based on the identified representative molecular structure.

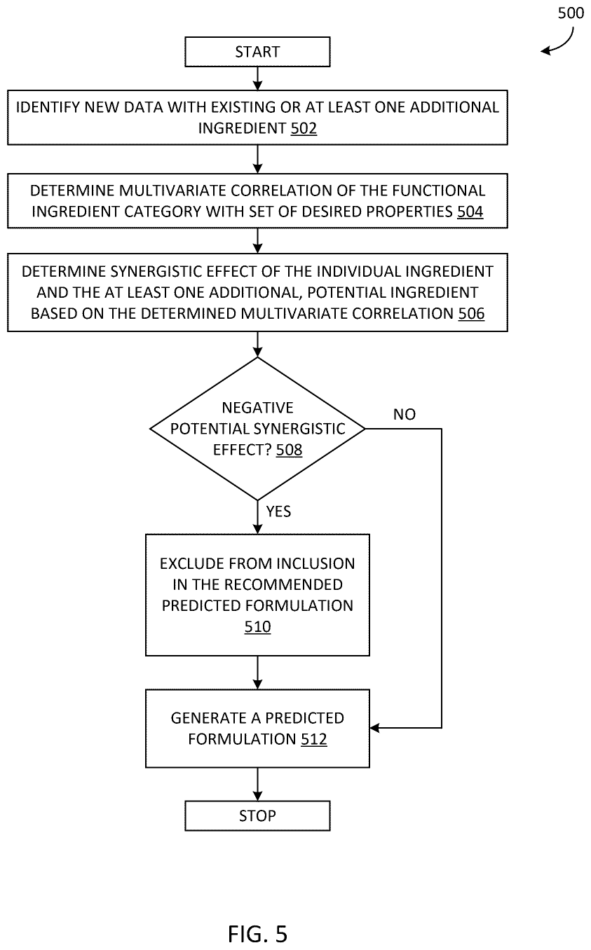

The recommendation generator 126 is an example of a ML or AI model that generates a recommended predicted formulation based on the generated set of desired properties. In some examples, the recommendation generator 126 is referred to as a fourth ML or AI model. The recommendation generator 126 implements a trained ML model to generate a recommendation that includes the recommended predicted formulation. As described herein, the recommended predicted formulation includes the identified representative molecular structure corresponding to the set of desired properties of the requested new formulation. The process by which the prediction generator 124 and recommendation generator 126 generate a predication and generate a recommendation, respectively, are described in greater detail below with regards to FIGS. 3A-3B and FIG. 5.

The data structure generator 128 is an example of a specialized processor, or processing unit, implemented on the molecule predictor 118 that generates a data structure that stores results generated by one or more of the category determiner 120, the structure identifier 122, the prediction generator 124, the recommendation generator 126, and the feedback receiver 130. In some examples, the generated data structure includes one or more of the determined set of INCI names, the associated set of desired properties, the extracted ingredient information, the determined functional ingredient category, the identified representative molecular structure of the individual ingredient, and the recommended predicted formulation as examples of data 116. The generated data structure is stored in the data storage device 114 and/or in external storage, such as on the server 132. The generated data structure is used to further and continuously train the ML or AI models of the molecule predictor 118, such as the category determiner 120, the structure identifier 122, the prediction generator 124, and the recommendation generator 126, in order to continue to provide improved results over time.

The feedback receiver 130 is an example of a specialized processor, or processing unit, implemented on the molecule predictor 118 that receives feedback on the generated recommendation of the predicted formulation. In some examples, the feedback is received via the user interface device 110. In other examples, the feedback is received from an external device 130 via the network 132. The received feedback is used to further and continuously train the ML or AI models of the molecule predictor 118, such as the category determiner 120, the structure identifier 122, the prediction generator 124, and the recommendation generator 126, in order to continue to provide improved results over time. In some examples, the data structure generator 128 writes received feedback into the generated data structure in order to store the received feedback as data 116.

The external device 132 is another example of a computing device, separate from and external to the computing device 102. In some examples, the external device 132 includes a mobile computing device or any other portable device. A mobile computing device includes, for example but without limitation, a mobile telephone, laptop, tablet, computing pad, netbook, gaming device, and/or portable media player. The external device 132 can also include less-portable devices such as servers, desktop personal computers, kiosks, or tabletop devices. Additionally, the external device 132 can represent a group of processing units or other computing devices. The server 134, in some examples, is an example of an external storage device, remote data storage device, a data storage in a remote data center, or a cloud storage.

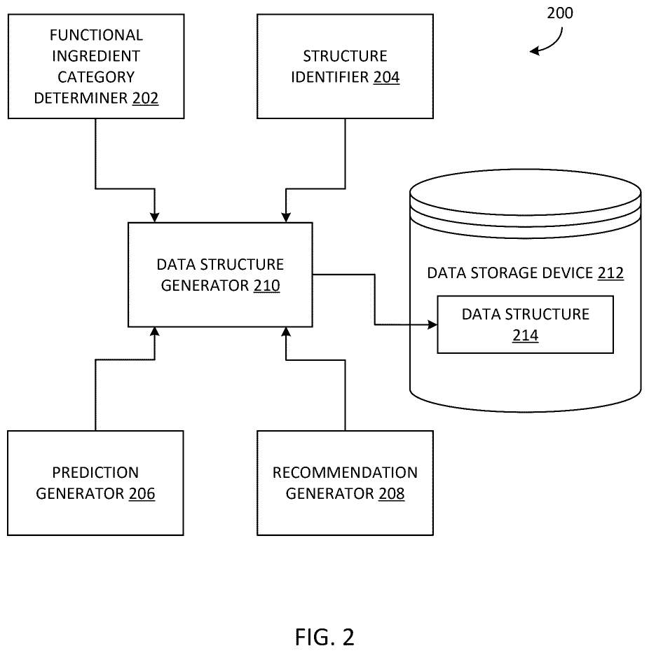

FIG. 2 illustrates an example system for determining a functional ingredient category, generating a prediction, and storing a generated data structure. The system 200 illustrated in FIG. 2 is provided for illustration only. Other examples of the system 200 can be used without departing from the scope of the present disclosure. In some examples, the system 200 augments formulation design according to one or more examples provided herein.

The system 200 illustrated in FIG. 2 includes a functional ingredient category determiner 202, a structure identifier 204, a prediction generator 206, a recommendation generator 208, a data structure generator 210, and a data storage device 212. In some examples, the functional ingredient category determiner 202 is an example of the category determiner 120, the structure identifier 204 is an example of the structure identifier 122, the prediction generator 206 is an example of the prediction generator 124, the recommendation generator 208 is an example of the recommendation generator 126, the data structure generator 210 is an example of the data structure generator 128, and the data storage device 212 is an example of the data storage device 114. As referenced herein, each of the functional ingredient category determiner 202, structure identifier 204, prediction generator 206, recommendation generator 208, and data structure generator 210 are implemented on the molecule predictor 118.

Upon receiving a request for a new formulation, the functional ingredient category determiner 202 determines a functional ingredient category for an individual ingredient, or multiple ingredients, in a requested new formulation. The structure identifier 204 identifies a representative molecular structure of the individual ingredient in the determined functional ingredient category, the prediction generator 206 implements a trained ML model to generate a predicted set of desired properties of the requested new formulation and of the identified representative molecular structure of the individual ingredient in the determined functional ingredient category based on the identified representative molecular structure, and the recommendation generator 208 implements a trained ML model to generate the recommended predicted formulation, that includes the identified representative molecular structure corresponding to the set of desired properties of the requested new formulation. The data structure generator 210 then generates a data structure 214 that stores results generated by one or more of the functional ingredient category determiner 202, the structure identifier 204, the prediction generator 206, and the recommendation generator 208. In some examples, the generated data structure includes one or more of the determined set of INCI names, the associated set of desired properties, the extracted ingredient information, the determined functional ingredient category, the identified representative molecular structure of the individual ingredient, and the recommended predicted formulation.

The generated data structure is stored in the data storage device 212 and/or in an external storage location, such as on the server 132. In some examples, the generated data structure 214 is a structured data structure that provides an improved mechanism for training and retraining the ML and AI models implemented by one or more of the functional ingredient category determiner 202, the structure identifier 204, the prediction generator 206, and the recommendation generator 208, as well as improves the facilitation of information retrieval by aggregating the outputs generated by the functional ingredient category determiner 202, the structure identifier 204, the prediction generator 206, and the recommendation generator 208, in addition to received feedback regarding generated recommendations.

FIGS. 3A-3B illustrate an example computer-implemented method of determining an ingredient functional category and generating a recommended predicted formula. The computer-implemented method 300 is presented for illustration only and should not be construed as limiting. Other examples of the computer-implemented method 300 can be used without departing from the scope of the present disclosure. The computer-implemented method 300 can be implemented by one or more electronic devices described herein, such as the computing device 102. It should be understood that FIG. 3B is a continuation of FIG. 3A and collectively, FIGS. 3A and 3B illustrate the computer-implemented method 300.

The computer-implemented method 300 begins by the computing device 102 receiving a request for a new formulation in operation 302. In some examples, the computing device 102 receives the request via the user interface device 110. In some examples, the computing device 102 receives the request from an external device, such as the external device 132, via the network 136. The received request identifies parameters that the requested new formulation is to meet. In various examples, the received request identifies one or more properties or characteristics the requested new formulation is to include, one or more examples of targeted property measurement results from the new or old formulations, ingredients to be specifically included or excluded in the requested new formulation, sustainability requirements for the requested new formulation, and so forth.

In operation 304, the category determiner 120 determines an ingredient in the requested formulation that matches a functional ingredient category list. In some examples, the category determiner 120 extracts a list, also referred to herein as a shortlist, of functional ingredient categories based on the parameters identified in the request from the data storage device 114 or the server 134. In some examples, the list is stored as an example of data 116 in the data storage device 114 and/or an external storage location such as the server 134. Each of the functional ingredient categories on the extracted list includes one or more select ingredients, such as a common active ingredient, in the functional ingredient category. The category determiner 120 determines an ingredient on the extracted list that matches the parameters in the requested new formulation. In some examples, the category determiner 120 makes a preliminary determination of the functional ingredient category as the category having the matching ingredient.

In operation 306, the structure identifier 122 identifies a molecular structure of an individual ingredient in the determined functional ingredient category. As referenced herein, the shortlist of functional ingredient categories is maintained in a database and stored in a storage location such as the data storage device 114 and/or the server 134 as an example of data 116. The database includes, in addition to functional ingredient categories and representative ingredients used in operation 304 to determine the ingredient in the requested new formulation that matches a functional ingredient category list, one or more molecular structures of individual ingredients. The structure identifier 122 utilizes the preliminary functional ingredient category and identified ingredient determined by the category determiner 120 and then extracts one or more molecular structures of one of the representative ingredients.

In operation 308, the structure identifier 122 inputs data associated with the identified molecular structure into the ML model used to generate a prediction of a set of desired properties for the requested new formulation. In some examples, the structure identifier 122 inputs the data associated with the identified molecular structure into the ML model without accompanying functional ingredient category data. It should be understood that operations 304-306 are the result of a sequential coarse-to-fine feature learning process to optimize the selection of chemical multilevel features for prediction of a set of desired properties for the requested new formulation. Though the learning operation is a sequential learning process, operation 308 operates through a batch inference process, enabling parallel processing of multiple data points associated with various formulations at one time.

In operation 310, the structure identifier 122 identifies, or generates, a prediction of a set of desired properties for the requested new formulation. In some examples, the prediction of the set of desired properties are included in the request, and in these examples operation 310 may be omitted. However, in examples where the prediction of the set of desired properties are not included in the request, the structure identifier 122 determines common properties of the representative ingredient having the identified molecular structures and, based on the determined common properties, generates the prediction of desired properties that a potential formulation with ingredients in the determined functional ingredient category and having the identified molecular structures are likely to have. It should be understood that, by the sequential, or hierarchical, determination of the ingredient in the functional category in operation 304 followed by the identification of the chemical characteristics of each molecular structure in the functional category in operation 306, the generation of the prediction of the set of desired properties in operation 310 accounts for the unique chemical characteristic of each ingredient within the same functional category that that are correlated to the targeted properties.

In some examples, the database is or includes a database of International Nomenclature Cosmetic Ingredient (INCI) names associated with the set of desired properties. The structure identifier 122 extracts, from a data source such as an additional database stored in the data storage device 114, ingredient information for the individual ingredient determined in operation 304, and, based on the extracted ingredient information, the functional ingredient category for the requested new formulation is determined.

In operation 312, the structure identifier 122 define constraints of ingredients to be used in the predicted formulation. In other words, the structure identifier 122 identifies and defines the parameters of the ingredients to be used and/or ingredients to explicitly not be used. In some examples, the defined constraints include, but are not limited to, sustainability constraints. For example, certain ingredients may be preferred or excluded based on a sustainability profile, manufacturing parameters, of the ingredients stored in the data storage device 114 as an example of data 116. In some examples, the data 116 includes a prioritized list of ingredients for a particular functional ingredient category that is organized based on the particular constraints. For example, the prioritized list may include an ordered priority of ingredients having a particular type of molecular structure or chemical characteristic(s) that meets the defined constraints. In some examples, the defined constraints are provided in the initially received request in operation 302. In some examples, the defined constraints are defined based on the data 116 stored in the data storage device 114.

In operation 314, the prediction generator 124 generates a recommended predicted formulation based on the generated prediction of the set of desired properties and the defined constraints of ingredients. The recommended predicted formulation includes the identified representative molecular structure corresponding to the set of desired properties of the requested new formulation. In other words, the recommended predicted formulation includes the properties desired of the requested new formulation and meets the defined constraints, or parameters, that may be specified in the initially received request, including but not limited to the inclusion or exclusion of particular ingredients, sustainability requirements, manufacturing parameters, and so forth.

In operation 316, the structure identifier 122 determines whether feedback regarding the labeled data has been received. Labeled data may be received from the category determiner 120, which implements a ML model as described herein and may determine whether the preliminary functional ingredient category matches the generated predicted set of desired properties. The category determiner 120 may label the generated prediction using a labeling system to identify whether the generated prediction is acceptable or not acceptable. For example, the labeling system may include updating the database to include the generated prediction and the identification as acceptable or not acceptable. In other examples, the labeled data may be received form an external device, such as the external device 132, that receives the generated prediction of the set of desired properties as a preliminary output from the molecule predictor 118. In examples where the labeled data is not received as feedback, the computer-implemented method 300 proceeds to operation 318 and transmits a request for labeled data as feedback. The request may be transmitted internally to the category determiner 120 or externally to the external device 132. Following the request being transmitted in operation 318, the computer-implemented method 300 returns to operation 316 and again determines whether the feedback has been received. In examples where the labeled data is received as feedback, the computer-implemented method 300 proceeds to operation 320.

In operation 320, the category determiner 120 determines the functional ingredient category of the labeled ingredients for the requested new formulation. In some examples, the category determiner 120 confirms the preliminary selection of a functional ingredient category. In some examples, the category determiner 120 determines the preliminary selection of the functional ingredient category is incorrect or not the most precise category and, based on the labeled data received as feedback, determines a different functional ingredient category is the most appropriate functional ingredient category for the requested new formulation. In other examples, where a preliminary selection was not previously made, the category determiner 120 determines the most appropriate functional ingredient category. In some examples, determining the functional ingredient category includes extracting in vitro and in vivo data from a database, such as the generated data structure 214 and/or recording the in vitro and in vivo data from a database, such as the generated data structure 214. The database may be stored on the data storage device 114 or on an external storage device, such as the server 134.

In operation 322, the category determiner 120 determines whether the functional ingredient category has been determined for each determined ingredient in the requested new formulation. A requested new formulation may include one or more than one ingredient from one or more functional categories. In other words, in various examples, the requested new formulation may be a request for one ingredient that is in one functional category, multiple ingredients from the same functional category, or multiple ingredients from multiple functional categories. In examples where the functional category of all ingredients has not been determined, the computer-implemented method 300 proceeds to operation 324 and triggers the determination, by the category determiner 120, of one or more additional functional ingredient categories. In examples where the functional category of all ingredients has been determined, the computer-implemented method 300 proceeds to operation 326.

In operation 326, the structure identifier 122 determines and evaluates any potential synergistic effects of the updated labeled data and the updated functional ingredient category data. As described in greater detail below, the structure identifier 122 determines a multivariate correlation of the functional ingredient category with the set of desired properties, which is used to measure the degree to which the properties included in the identified or generated set of desired properties described herein of a particular, individual ingredient may be affected by the presence of one or more additional, potential ingredients that may also be present in a formulation.

In operation 328, the data structure generator 128 updates the list of functional ingredient categories based at least in part on the determined potential synergistic effects determined in operation 326. As referenced herein, the shortlist of functional ingredient categories is maintained in a database and stored in a storage location such as the data storage device 114 and/or the server 134, as an example of data 116. In some examples, the database is referred to as an ingredient information library. Based on the labeled data received as feedback and the determined functional ingredient category of the labeled ingredients, the data structure generator 128 updates the shortlist to be used for future extraction of an ingredient, determination of a functional ingredient category for a new request, and retraining of one or more ML models implemented by the category determiner 120 and the structure identifier 122.

In operation 330, the category determiner 120 determines whether an update, or retraining, of the ML model used for determining a functional ingredient category is needed based on newly available data, including the updated, labeled data received as feedback, the determined functional ingredient category of the labeled ingredients, and the updated shortlist. In some examples, an update or retraining is determined to be completed based on any new data being available, such as feedback being received. For example, the category determiner 120 updates the functional ingredient category or categories associated with the particular ingredients and molecular structures. In examples where an update is determined to be needed, the computer-implemented method 300 returns to operation 304. In examples where the update is determined to not be needed, the computer-implemented method 300 proceeds to operation 332.

In operation 332, the structure identifier 122 determines whether an update, or retraining, of the ML model used for updating the molecular structure, or structures, of the updated ingredient in the determined functional ingredient category used to determining a molecular structure and predicting a set of desired properties is needed based on newly available data, including the updated, labeled data received as feedback, the determined functional ingredient category of the labeled ingredients, and the updated shortlist. In some examples, an update or retraining is determined to be completed based on any new data being available, such as feedback being received. For example, the structure identifier 122 updates the molecular structures of the updated ingredients in the determined functional ingredient category. In examples where an update is determined to be needed, the computer-implemented method 300 returns to operation 308. In examples where the update is determined to not be needed, the computer-implemented method 300 proceeds to operation 334.

Accordingly, through at least operations 330 and 332 the ML models implemented by the molecule predictor 118 are continuously batch retrained and improving in order to continuously provide improved determinations and recommendations. However, in some examples operations 330 and 332 may be omitted and not included in the computer-implemented method 300. For example, the computer-implemented method 300 may be implemented on a local device and deployed independently, such that continuous retraining is not feasible. In that instance, operation 330 may be omitted to be performed at a later time.

In operation 334, the recommendation generator generates an output that includes the previously generated recommended formulation as well as additional information associated with the generated predicted formulation. In some examples, the additional information may include, but is not limited to, one or more of initially predicted properties of the recommended formulation, actual properties of the recommended formulation, the updated ML model, additional analysis of the requested new formulation, parameters included in the request, ingredient characteristics, the determined functional ingredient category or categories, potential alternative functional ingredient categories or ingredients, and challenges that were overcome in creating the generated predicted formulation. For example, the recommendation may include details about alternative ingredients that could be used but may be more difficult to source or manufacture, were not included due to potential or known negative interactions with other ingredients in the formulation, or were not selected due to weaker test results or sustainability ratings. The updated ML model serves to save a copy of the generated data, for example as data 116 on the data storage device 114 and/or external storage such as the server 134, as well as the output in response to the initially received request for a recommended formulation.

The generated recommendation is output for review as the response to the received request for the new formulation. In some examples, the generated recommendation is output on the computing device 102, such as via the user interface device 110. In some examples, the generated recommendation is output to an external device, such as the external device 132, via the communications interface device 112 and the network 136. Following the output of the generated recommendation, the computer-implemented method 300 terminates.

In some examples, the feedback receiver 130 determines whether feedback is received in response to the output generated recommendation. For example, feedback may be received that indicates an acceptance of the generated predicted formulation, as a request for additional formulations instead of or in addition to the generated predicted formulation, as a request for additional information regarding how the generated predicted formulation was generated, as a request for additional information regarding the generated predicted formulation, and so forth. In examples where feedback is received, the category determiner 120 and the structure identifier 122 update, or retrain, their respective ML models based on the received feedback. In some examples, this may trigger a reinitiation of the computer-implemented method 300 to generate a new predicted formulation by determining an ingredient in the requested formulation that matches a functional ingredient category list.

FIG. 4 illustrates an example computer-implemented method of determining a functional ingredient category. The computer-implemented method 400 is presented for illustration only and should not be construed as limiting. Other examples of the computer-implemented method 400 can be used without departing from the scope of the present disclosure. The computer-implemented method 400 can be implemented by one or more electronic devices described herein, such as the computing device 102.

The computer-implemented method 400 begins by the structure identifier 122 identifying, or generating, a set of desired properties for the requested new formulation in operation 402. As referenced herein, such as in operation 308 of the computer-implemented method 300, in some examples, the set of desired properties are included in the request, and in these examples operation 308 may be omitted. However, in examples where desired properties are not included in the request, the structure identifier 122 determines common properties of the representative ingredient having the identified molecular structures and, based on the determined common properties, generates a prediction of desired properties that a potential formulation with ingredients in the determined functional ingredient category and having the identified molecular structures are likely to have.

In operation 404, the structure identifier 122 determines one or both of a set of INCI names or a Chemical Abstracts Service (CAS) number associated with the set of desired properties. As referenced herein, the structure identifier 122 determines the set of INCI names that meet the desired properties based on the database stored in the data storage device 114 and/or the server 134.

In operation 406, the structure identifier 122 extracts ingredient information for the representative ingredients. In some examples, the extracted ingredient information includes a recommendation of a functional ingredient category for a representative ingredient. In some examples, the structure identifier 122 extracts and presents publicly available ingredient information for the representative ingredient or ingredients. In some examples, the structure identifier 122 extracts and presents proprietary and internal ingredient information, including previously labeled functional ingredient category for the representative ingredient. In some examples, the structure identifier 122 extracts and presents both publicly available and proprietary ingredient information.

In operation 408, the category determiner 120 determines the functional ingredient category based on the identified set of desired properties, determined set of INCI names, and extracted ingredient information. As referenced herein, in some examples, the category determiner 120 confirms the preliminary selection of a functional ingredient category. In some examples, the category determiner 120 determines the preliminary selection of the functional ingredient category is incorrect or not the most precise category and, based on the labeled data received as feedback, determines a different functional ingredient category is the most appropriate functional ingredient category for the requested new formulation. In other examples, where a preliminary selection was not previously made, the category determiner 120 determines the most appropriate functional ingredient category.

In operation 410, the category determiner 120 determines whether the determination of each functional ingredient category is complete. For example, as in operation 316, the requested new formulation may include one or more than one ingredient from one or more functional categories. In examples where the functional category of all ingredients has not been determined, the computer-implemented method 400 returns to operation 406 and the structure identifier 122 extracts additional ingredient information. In examples where the functional category of all ingredients has been determined, the computer-implemented method 400 proceeds to operation 412.

In operation 412, the database generator 128 updates the ingredient information library. As referenced herein, the ingredient information library is a database, stored as an example of data 116 on the data storage device 114 and/or the server 134, that maintains the shortlist of functional ingredient categories and associated ingredients, ingredient characteristics, and so forth. The database generator 128 updates the ingredient information library based on the determined functional ingredient category, or categories, determined in operation 408.

In operation 414, the database generator 128 generates, if not already created, or updates, if previously created, a data structure, such as the data structure 214. The data structure stores results generated by one or more of the functional ingredient category determiner 202, the structure identifier 204, the prediction generator 206, and the recommendation generator 208. In some examples, the generated data structure includes one or more of the determined set of INCI names, the associated set of desired properties, the extracted ingredient information, the determined functional ingredient category, the identified representative molecular structure of the individual ingredient, and the recommended predicted formulation. As referenced herein, the generated data structure facilitates more efficient and effective information retrieval than existing structures. Following operation 414, the computer-implemented method 400 terminates.

FIG. 5 illustrates an example computer-implemented method of generating a recommended predicted formula. The computer-implemented method 500 is presented for illustration only and should not be construed as limiting. Other examples of the computer-implemented method 500 can be used without departing from the scope of the present disclosure. The computer-implemented method 500 can be implemented by one or more electronic devices described herein, such as the computing device 102.

The computer-implemented method 500 begins by the structure identifier 122 identifying new data with an existing or at least one additional ingredient in operation 502. For example, the structure identifier 122 identifies new data associated with an existing ingredient or data from at least one additional ingredient related to the functional ingredient category. In some examples, the new data is identified based on an alert being received that a data structure, such as the generated data structure 214 or another source of data 116 stored in the data storage device 114 or the server 134 is updated to include new information. In some examples, the new data is identified based on a periodic scan of the data structure 214 or other source of data 116 that is performed at a regular, periodic interval, such as every twelve hours, once a day, once every two days, once a week, and so forth.

In operation 504, the structure identifier 122 determines a multivariate correlation of the functional ingredient category with the set of desired properties. The determined multivariate correlation measures the degree to which a particular variable may be predicted based on other variables. Accordingly, the determined multivariate correlation measures the degree to which the properties included in the identified or generated set of desired properties described herein of a particular, individual ingredient may be affected by the presence of one or more additional, potential ingredients that may also be present in a formulation. For example, the characteristics of a particular molecule may be enhanced, reduced, eliminated, or unaffected due to the additional of an additional molecule. By determining the multivariate correlation, this potentially synergistic effect may be estimated.

In operation 506, the structure identifier 122 determines the synergistic effect of an individual ingredient and the at least one additional, potential ingredient based on the determined multivariate correlation. For example, the measurement of the degree of changes in the accuracy rate when a functional category is removed may be used to determine the potential synergistic effect of the individual ingredient and the at least one additional, potential ingredient.

In operation 508, the prediction generator 124 determines whether the determined potential synergistic effect between an individual, or representative, ingredient is negative. For example, a negative synergistic effect is one that eliminates or reduces the efficacy of a particular ingredient, or molecule, that otherwise may be present and included in a recommended predicted formulation. In examples where a negative potential synergistic effect is determined, the computer-implemented method 500 proceeds to operation 510 and excludes the additional ingredient or the functional ingredient category from inclusion in the recommended, predicted formulation. The degree of exclusion, i.e., whether the additional ingredient alone or the entire functional ingredient category to which the additional ingredient belongs, is dependent on the determination in operation 506. Where the determination is one that indicates the additional ingredient in particular would produce the negative synergistic effect, and such as negative synergistic effect is not extrapolated to all ingredients in the functional ingredient category, only additional ingredient is excluded. However, where the determination is one that indicates the characteristics of the functional ingredient category as a whole would produce a negative synergistic effect, the entire functional ingredient category is excluded from inclusion in the recommended predicted formulation.

Following operation 510, and in examples of operation 508 where a negative potential synergistic effect is not determined, the computer-implemented method 500 proceeds to operation 512 and generates the predicted formulation. As referenced herein, the predicted formulation includes the properties desired of the requested new formulation and meets any of the additional parameters that may be specified in the initially received request, including but not limited to the inclusion or exclusion of particular ingredients, sustainability requirements, manufacturing parameters, and so forth. In examples where a potential negative synergistic effect is determined in operation 508, the ingredient or functional ingredient category is excluded, and therefore not included in, the generated predicted formulation. Following operation 512, the computer-implemented method 500 terminates.

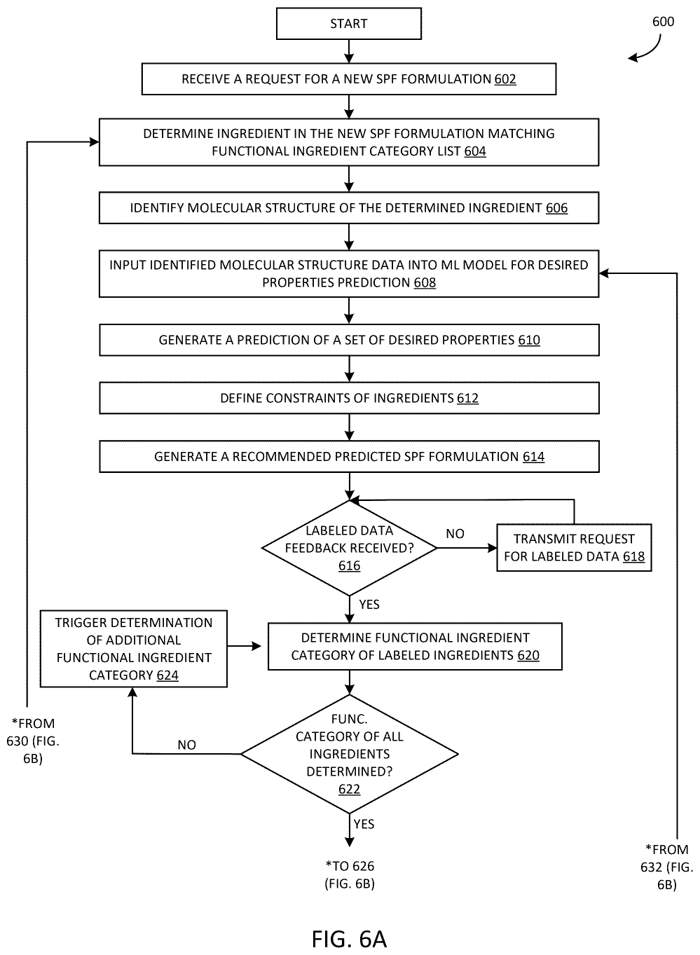

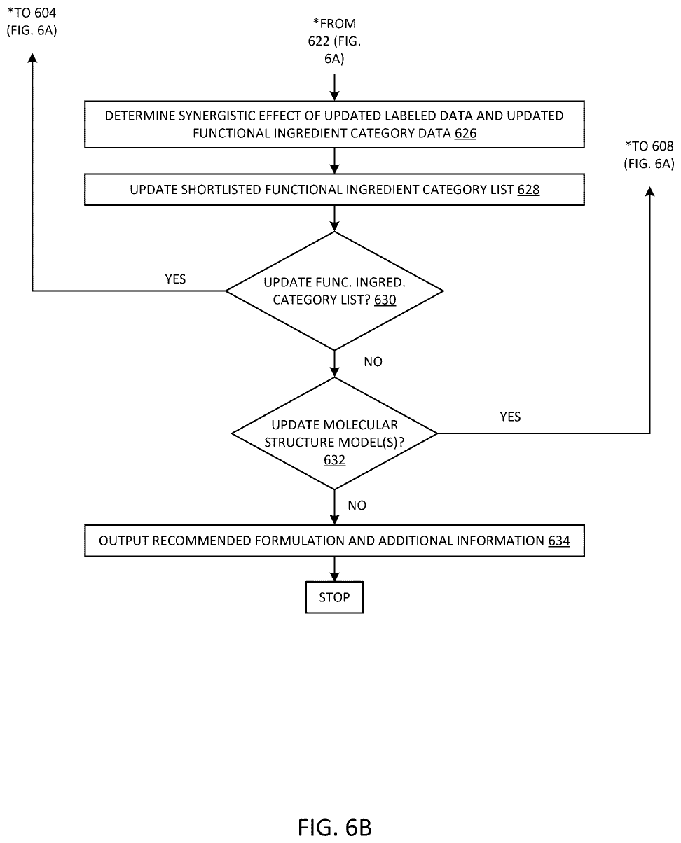

FIGS. 6A-6B illustrate an example computer-implemented method of generating a recommended predicted formula having a requested sunscreen protection factor (SPF). The computer-implemented method 600 is presented for illustration only and should not be construed as limiting. Other examples of the computer-implemented method 600 can be used without departing from the scope of the present disclosure. The computer-implemented method 600 can be implemented by one or more electronic devices described herein, such as the computing device 102. It should be understood that FIG. 6B is a continuation of FIG. 6A and collectively, FIGS. 6A and 6B illustrate the computer-implemented method 600.

The computer-implemented method 600 begins by receiving a request for a new sunscreen protection factor (SPF) formulation in operation 602. In some examples, the computing device 102 receives the request via the user interface device 110. In some examples, the computing device 102 receives the request from an external device, such as the external device 132, via the network 136. The received request identifies parameters that the requested new SPF formulation is to meet. In various examples, the received request identifies one or more SPF properties or characteristics the requested new SPF formulation is to include, one or more examples of targeted property measurement results including from in vitro and in vivo tests from the new or old SPF formulations is to meet, ingredients to be specifically included or excluded in the requested new SPF formulation, sustainability requirements for the requested new SPF formulation, and so forth.

In operation 604, the category determiner 120 determines an ingredient in the requested SPF formulation that matches a functional ingredient category list. In some examples, the category determiner 120 extracts a list, also referred to herein as a shortlist, of functional ingredient categories based on the parameters identified in the request from the data storage device 114 or the server 134. In some examples, the list is stored as an example of data 116 in the data storage device 114 and/or an external storage location such as the server 134. Each of the functional ingredient categories on the extracted list includes one or more select ingredients, such as a common active ingredient, in the functional ingredient category. The category determiner 120 determines an ingredient on the extracted list that matches the parameters in the requested new SPF formulation. In some examples, the category determiner 120 makes a preliminary determination of the functional ingredient category as the category having the matching ingredient.

In operation 606, the structure identifier 122 identifies a molecular structure of an individual ingredient in the determined functional ingredient category. As referenced herein, the shortlist of functional ingredient categories is maintained in a database and stored in a storage location such as the data storage device 114 and/or the server 134 as an example of data 116. The database includes, in addition to functional ingredient categories and representative ingredients used in operation 604 to determine the ingredient in the requested new SPF formulation that matches a functional ingredient category list, one or more molecular structures of individual ingredients. The structure identifier 122 utilizes the preliminary functional ingredient category and identified ingredient determined by the category determiner 120 and then extracts one or more molecular structures of one of the representative ingredients.

In operation 608, the structure identifier 122 inputs data associated with the identified molecular structure into the ML model used to generate a prediction of a set of desired properties for the requested new SPF formulation. In some examples, the structure identifier 122 inputs the data associated with the identified molecular structure into the ML model without accompanying functional ingredient category data. It should be understood that this enables the molecular predictor 118 to operate through a batch machine learning process, rather than strictly as an online learning process. It should be understood that operations 604-606 are the result of a sequential coarse-to-fine feature learning process to optimize the selection of chemical multilevel features for prediction of a set of desired properties for the requested new formulation. Though the learning operation is a sequential learning process, operation 608 operates through a batch inference process, enabling parallel processing of multiple data points associated with various formulations at one time.

In operation 610, the structure identifier 122 identifies, or generates, a prediction of a set of desired properties for the requested new SPF formulation. In some examples, the prediction of the set of desired properties are included in the request, and in these examples operation 610 may be omitted. However, in examples where the prediction of the set of desired properties are not included in the request, the structure identifier 122 determines common properties of the representative ingredient having the identified molecular structures and, based on the determined common properties, generates the prediction of desired properties that a potential SPF formulation with ingredients in the determined functional ingredient category and having the identified molecular structures are likely to have. It should be understood that, by the sequential, or hierarchical, determination of the ingredient in the functional category in operation 604 followed by the identification of the chemical characteristics of each molecular structure in the functional category in operation 606, the generation of the prediction of the set of desired properties in operation 610 accounts for the unique chemical characteristic of each ingredient within the same functional category that that are correlated to the targeted properties.

In some examples, the database is or includes a database of INCI names associated with the set of desired properties. The structure identifier 122 extracts, from a data source such as an additional database stored in the data storage device 114, ingredient information for the individual ingredient determined in operation 604, and, based on the extracted ingredient information, the functional ingredient category for the requested new SPF formulation is determined.

In operation 612, the structure identifier 122 define constraints of ingredients to be used in the predicted SPF formulation. In other words, the structure identifier 122 identifies and defines the parameters of the ingredients to be used and/or ingredients to explicitly not be used. In some examples, the defined constraints include, but are not limited to, sustainability constraints. For example, certain ingredients may be preferred or excluded based on a sustainability profile, manufacturing parameters, of the ingredients stored in the data storage device 114 as an example of data 116. In some examples, the data 116 includes a prioritized list of ingredients for a particular functional ingredient category that is organized based on the particular constraints. For example, the prioritized list may include an ordered priority of ingredients having a particular type of molecular structure or chemical characteristic(s) that meets the defined constraints. In some examples, the defined constraints are provided in the initially received request in operation 602. In some examples, the defined constraints are defined based on the data 116 stored in the data storage device 114.

In operation 614, the prediction generator 124 generates a recommended predicted SPF formulation based on the generated prediction of the set of desired properties and the defined constraints of ingredients. The recommended predicted SPF formulation includes the identified representative molecular structure corresponding to the set of desired properties of the requested new SPF formulation. In other words, the recommended predicted SPF formulation includes the properties desired of the requested new SPF formulation and meets the defined constraints, or parameters, that may be specified in the initially received request, including but not limited to the inclusion or exclusion of particular ingredients, sustainability requirements, manufacturing parameters, and so forth.

In operation 616, the structure identifier 122 determines whether feedback regarding the labeled data has been received. In some examples, the labeled data includes in vivo, i.e., clinical, data and/or in vitro labeled data. Labeled data may be received from the category determiner 120, which implements a ML model as described herein and may determine whether the preliminary functional ingredient category matches the generated predicted set of desired properties. The category determiner 120 may label the generated prediction using a labeling system to identify whether the generated prediction is acceptable or not acceptable. For example, the labeling system may include updating the database to include the generated prediction and the identification as acceptable or not acceptable. In other examples, the labeled data may be received form an external device, such as the external device 132, that receives the generated prediction of the set of desired properties as a preliminary output from the molecule predictor 118. In examples where the labeled data is not received as feedback, the computer-implemented method 600 proceeds to operation 618 and transmits a request for labeled data as feedback. The request may be transmitted internally to the category determiner 120 or externally to the external device 132. Following the request being transmitted in operation 618, the computer-implemented method 600 returns to operation 616 and again determines whether the feedback has been received. In examples where the labeled data is received as feedback, the computer-implemented method 600 proceeds to operation 620.

In operation 620, the category determiner 120 determines the functional ingredient category of the labeled ingredients for the requested new SPF formulation. In some examples, the category determiner 120 confirms the preliminary selection of a functional ingredient category. In some examples, the category determiner 120 determines the preliminary selection of the functional ingredient category is incorrect or not the most precise category and, based on the labeled data received as feedback, determines a different functional ingredient category is the most appropriate functional ingredient category for the requested new SPF formulation. In other examples, where a preliminary selection was not previously made, the category determiner 120 determines the most appropriate functional ingredient category. In some examples, determining the functional ingredient category includes extracting in vitro and in vivo SPF data from a database, such as the generated data structure 214 and/or recording the in vitro and in vivo SPF data from a database, such as the generated data structure 214. The database may be stored on the data storage device 114 or on an external storage device, such as the server 134.

In operation 622, the category determiner 120 determines whether the functional ingredient category has been determined for each determined ingredient in the requested new SPF formulation. A requested new SPF formulation may include one or more than one ingredient from one or more functional categories. In other words, in various examples, the requested new SPF formulation may be a request for one ingredient that is in one functional category, multiple ingredients from the same functional category, or multiple ingredients from multiple functional categories. In examples where the functional category of all ingredients has not been determined, the computer-implemented method 600 proceeds to operation 624 and triggers the determination, by the category determiner 120, of one or more additional functional ingredient categories. In examples where the functional category of all ingredients has been determined, the computer-implemented method 600 proceeds to operation 626.

In operation 626, the structure identifier 122 determines and evaluates any potential synergistic effects of the updated labeled data and the updated functional ingredient category data. As described in greater detail below, the structure identifier 122 determines a multivariate correlation of the functional ingredient category with the set of desired properties, which is used to measure the degree to which the properties included in the identified or generated set of desired properties described herein of a particular, individual ingredient may be affected by the presence of one or more additional, potential ingredients that may also be present in an SPF formulation.

In operation 628, the data structure generator 128 updates the list of functional ingredient categories based at least in part on the determined potential synergistic effects determined in operation 626. As referenced herein, the shortlist of functional ingredient categories is maintained in a database and stored in a storage location such as the data storage device 114 and/or the server 134, as an example of data 116. In some examples, the database is referred to as an ingredient information library. Based on the labeled data received as feedback and the determined functional ingredient category of the labeled ingredients, the data structure generator 128 updates the shortlist to be used for future extraction of an ingredient, determination of a functional ingredient category for a new request, and retraining of one or more ML models implemented by the category determiner 120 and the structure identifier 122.

In operation 630, the category determiner 120 determines whether an update, or retraining, of the ML model used for determining a functional ingredient category is needed based on newly available data, including the updated, labeled data received as feedback, the determined functional ingredient category of the labeled ingredients, and the updated shortlist. In some examples, an update or retraining is determined to be completed based on any new data being available, such as feedback being received. For example, the category determiner 120 updates the functional ingredient category or categories associated with the particular ingredients and molecular structures. In examples where an update is determined to be needed, the computer-implemented method 600 returns to operation 604. In examples where the update is determined to not be needed, the computer-implemented method 600 proceeds to operation 632.

In operation 632, the structure identifier 122 determines whether an update, or retraining, of the ML model used for updating the molecular structure, or structures, of the updated ingredient in the determined functional ingredient category used to determining a molecular structure and predicting a set of desired properties is needed based on newly available data, including the updated, labeled data received as feedback, the determined functional ingredient category of the labeled ingredients, and the updated shortlist. In some examples, an update or retraining is determined to be completed based on any new data being available, such as feedback being received. For example, the structure identifier 122 updates the molecular structures of the updated ingredients in the determined functional ingredient category. In examples where an update is determined to be needed, the computer-implemented method 600 returns to operation 608. In examples where the update is determined to not be needed, the computer-implemented method 600 proceeds to operation 634.

Accordingly, through at least operations 630 and 632 the ML models implemented by the molecule predictor 118 are continuously batch retrained and improving in order to continuously provide improved determinations and recommendations. However, in some examples operations 630 and 632 may be omitted and not included in the computer-implemented method 600. For example, the computer-implemented method 600 may be implemented on a local device and deployed independently, such that continuous retraining is not feasible. In that instance, operation 630 may be omitted to be performed at a later time.

In operation 634, the recommendation generator generates an output that includes the previously generated recommended SPF formulation as well as additional information associated with the generated predicted SPF formulation. In some examples, the additional information may include, but is not limited to, one or more of initially predicted properties of the recommended SPF formulation, actual properties of the recommended SPF formulation, the updated ML model, additional analysis of the requested new SPF formulation, parameters included in the request, ingredient characteristics, the determined functional ingredient category or categories, potential alternative functional ingredient categories or ingredients, and challenges that were overcome in creating the generated predicted SPF formulation. For example, the recommendation may include details about alternative ingredients that could be used but may be more difficult to source or manufacture, were not included due to potential or known negative interactions with other ingredients in the SPF formulation, or were not selected due to weaker test results or sustainability ratings. The updated ML model serves to save a copy of the generated data, for example as data 116 on the data storage device 114 and/or external storage such as the server 134, as well as the output in response to the initially received request for a recommended formulation.

The generated recommendation is output for review as the response to the received request for the new SPF formulation. In some examples, the generated recommendation is output on the computing device 102, such as via the user interface device 110. In some examples, the generated recommendation is output to an external device, such as the external device 132, via the communications interface device 112 and the network 136. Following the output of the generated recommendation, the computer-implemented method 600 terminates.

In some examples, the feedback receiver 130 determines whether feedback is received in response to the output generated recommendation. For example, feedback may be received that indicates an acceptance of the generated predicted SPF formulation, as a request for additional SPF formulations instead of or in addition to the generated predicted SPF formulation, as a request for additional information regarding how the generated predicted SPF formulation was generated, as a request for additional information regarding the generated predicted SPF formulation, and so forth. In examples where feedback is received, the category determiner 120 and the structure identifier 122 update, or retrain, their respective ML models based on the received feedback. In some examples, this may trigger a reinitiation of the computer-implemented method 600 to generate a new predicted SPF formulation by determining an ingredient in the requested SPF formulation that matches a functional ingredient category list.

### Example Operating Environment

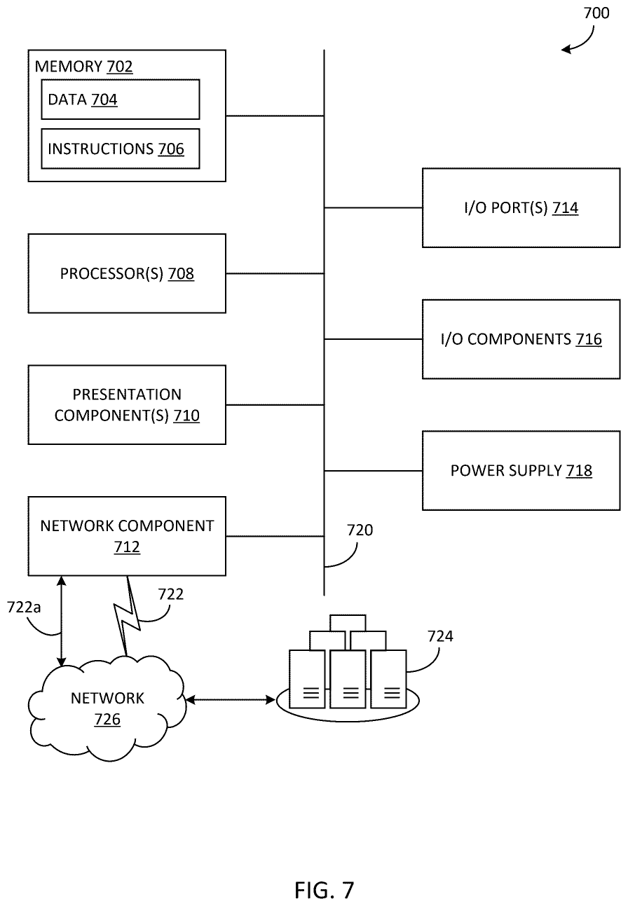

FIG. 7 is a block diagram of an example computing device 700 for implementing aspects disclosed herein and is designated generally as computing device 700. Computing device 700 is an example of a suitable computing environment and is not intended to suggest any limitation as to the scope of use or functionality of the examples disclosed herein. Neither should computing device 700 be interpreted as having any dependency or requirement relating to any one or combination of components/modules illustrated. The examples disclosed herein may be described in the general context of computer code or machine-useable instructions, including computer-executable instructions such as program components, being executed by a computer or other machine, such as a personal data assistant or other handheld device. Generally, program components including routines, programs, objects, components, data structures, and the like, refer to code that performs particular tasks, or implement particular abstract data types. The disclosed examples may be practiced in a variety of system configurations, including personal computers, laptops, smart phones, mobile tablets, hand-held devices, consumer electronics, specialty computing devices, etc. The disclosed examples may also be practiced in distributed computing environments when tasks are performed by remote-processing devices that are linked through a communications network.

Computing device 700 includes a bus 720 that directly or indirectly couples the following devices: computer-storage memory 702, one or more processors 708, one or more presentation components 710, I/O ports 714, I/O components 716, a power supply 718, and a network component 712. While computing device 700 is depicted as a seemingly single device, multiple computing devices 700 may work together and share the depicted device resources. For example, memory 702 may be distributed across multiple devices, and processor(s) 708 may be housed with different devices.

Bus 720 represents what may be one or more busses (such as an address bus, data bus, or a combination thereof). Although the various blocks of FIG. 7 are shown with lines for the sake of clarity, delineating various components may be accomplished with alternative representations. For example, a presentation component such as a display device is an I/O component in some examples, and some examples of processors have their own memory. Distinction is not made between such categories as “workstation,” “server,” “laptop,” “hand-held device,” etc., as all are contemplated within the scope of FIG. 7 and the references herein to a “computing device.” Memory 702 may take the form of the computer-storage media references below and operatively provide storage of computer-readable instructions, data structures, program modules and other data for computing device 700. In some examples, memory 702 stores one or more of an operating system, a universal application platform, or other program modules and program data. Memory 702 is thus able to store and access data 704 and instructions 706 that are executable by processor 708 and configured to carry out the various operations disclosed herein.

In some examples, memory 702 includes computer-storage media in the form of volatile and/or nonvolatile memory, removable or non-removable memory, data disks in virtual environments, or a combination thereof. Memory 702 may include any quantity of memory associated with or accessible by computing device 700. Memory 702 may be internal to computing device 700 (as shown in FIG. 7), external to computing device 700, or both. Examples of memory 702 include, without limitation, random access memory (RAM); read only memory (ROM); electronically erasable programmable read only memory (EEPROM); flash memory or other memory technologies; CD-ROM, digital versatile disks (DVDs) or other optical or holographic media; magnetic cassettes, magnetic tape, magnetic disk storage or other magnetic storage devices; memory wired into an analog computing device; or any other medium for encoding desired information and for access by computing device 700. Additionally, or alternatively, memory 702 may be distributed across multiple computing devices 700, for example, in a virtualized environment in which instruction processing is carried out on multiple computing devices 700. For the purposes of this disclosure, “computer storage media,” “computer-storage memory,” “memory,” and “memory devices” are synonymous terms for computer-storage memory 702, and none of these terms include carrier waves or propagating signaling.

Processor(s) 708 may include any quantity of processing units that read data from various entities, such as memory 702 or I/O components 716 and may include CPUs and/or GPUs. Specifically, processor(s) 708 are programmed to execute computer-executable instructions for implementing aspects of the disclosure. The instructions may be performed by the processor, by multiple processors within computing device 700, or by a processor external to client computing device 700. In some examples, processor(s) 708 are programmed to execute instructions such as those illustrated in the in the accompanying drawings. Moreover, in some examples, processor(s) 708 represent an implementation of analog techniques to perform the operations described herein. For example, the operations may be performed by an analog client computing device 700 and/or a digital client computing device 700. Presentation component(s) 710 present data indications to a user or other device. Exemplary presentation components include a display device, speaker, printing component, vibrating component, etc. One skilled in the art will understand and appreciate that computer data may be presented in a number of ways, such as visually in a graphical user interface (GUI), audibly through speakers, wirelessly between computing devices 700, across a wired connection, or in other ways. I/O ports 714 allow computing device 700 to be logically coupled to other devices including I/O components 716, some of which may be built in. Example I/O components 716 include, for example but without limitation, a microphone, joystick, game pad, satellite dish, scanner, printer, wireless device, etc.

Computing device 700 may operate in a networked environment via network component 712 using logical connections to one or more remote computers. In some examples, network component 712 includes a network interface card and/or computer-executable instructions (e.g., a driver) for operating the network interface card. Communication between computing device 700 and other devices may occur using any protocol or mechanism over any wired or wireless connection. In some examples, network component 712 is operable to communicate data over public, private, or hybrid (public and private) using a transfer protocol, between devices wirelessly using short range communication technologies (e.g., near-field communication (NFC), Bluetooth™ branded communications, or the like), or a combination thereof. Network component 712 communicates over wireless communication link 722 and/or a wired communication link 722a to a cloud resource 724 across network 726. Various different examples of communication links 722 and 722a include a wireless connection, a wired connection, and/or a dedicated link, and in some examples, at least a portion is routed through the internet.

Although described in connection with an example computing device 700, examples of the disclosure are capable of implementation with numerous other general-purpose or special-purpose computing system environments, configurations, or devices. Examples of well-known computing systems, environments, and/or configurations that may be suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, virtual reality (VR) devices, augmented reality (AR) devices, mixed reality devices, holographic device, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other examples of the disclosure may include different computer-executable instructions or components having more or less functionality than illustrated and described herein. In examples involving a general-purpose computer, aspects of the disclosure transform the general-purpose computer into a special-purpose computing device when configured to execute the instructions described herein.

By way of example and not limitation, computer readable media comprise computer storage media and communication media. Computer storage media include volatile and nonvolatile, removable and non-removable memory implemented in any method or technology for storage of information such as computer readable instructions, data structures, program modules, or the like. Computer storage media are tangible and mutually exclusive to communication media. Computer storage media are implemented in hardware and are non-transitory, i.e., exclude carrier waves and propagated signals. Computer storage media for purposes of this disclosure are not signals per se. Exemplary computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium that may be used to store information for access by a computing device. In contrast, communication media typically embody computer readable instructions, data structures, program modules, or the like in a modulated data signal such as a carrier wave or other transport mechanism and include any information delivery media.

In some examples, a computer-implemented method is provided. The method includes receiving a request for a new formulation having a set of desired properties; determining a functional ingredient category for an individual ingredient in the requested new formulation; identifying a representative molecular structure of the individual ingredient in the determined functional ingredient category; generating, by a trained machine learning (ML) model, a predicted set of desired properties of i) the requested new formulation and ii) the identified representative molecular structure of the individual ingredient in the determined functional ingredient category; and generating, based on the generated predicted set of desired properties, a recommended predicted formulation, the predicted formulation including the identified representative molecular structure corresponding to the set of desired properties of the requested new formulation.

In some examples, a system is provided. The system includes a memory, a transceiver to receive a request for a new formulation having a set of desired properties, and a processor coupled to the memory. The processor is configured to determine a functional ingredient category for an individual ingredient in the requested new formulation; identify a representative molecular structure of the individual ingredient in the determined functional ingredient category; generate, by a trained machine learning (ML), a predicted set of desired properties of i) the requested new formulation and ii) the identified representative molecular structure of the individual ingredient in the determined functional ingredient category; and generate, based on the generated predicted set of desired properties, a recommended predicted formulation, the recommended predicted formulation including the identified representative molecular structure corresponding to the set of desired properties of the requested new formulation.

In some examples, a computer-readable storage media is provided. The computer-readable storage media stores instructions that, when executed by a processor, cause the processor to control to receive a request for a new sunscreen protection factor (SPF) formulation having a set of desired properties; determine a functional ingredient category for an individual ingredient in the requested new formulation, wherein the functional ingredient category includes at least one of an SPF value, a UVA filter, or a UVB filter; identify a representative molecular structure of the individual ingredient in the determined functional ingredient category; generate, by a trained machine learning (ML), a predicted set of desired properties of i) the requested new SPF formulation and ii) the identified representative molecular structure of the individual ingredient in the determined functional ingredient category; and generate, based on the generated predicted set of desired properties, a recommended predicted SPF formulation, the recommended predicted SPF formulation including the identified representative molecular structure corresponding to the set of desired properties of the requested new SPF formulation.

Further examples are further described herein.

Various examples further include one or more of the following:
determining a set of nomenclature names associated with the set of desired properties, the set of nomenclature names being the individual ingredient, wherein the set of nomenclature names includes at least one of an International Nomenclature Cosmetic Ingredient (INCI) or a Chemical Abstracts Service (CAS) numbers;
extracting, from a data source, ingredient information for the individual ingredient;
based on the extracted ingredient information, determining the functional ingredient category for the requested new formulation;
generating a data structure comprising the determined set of nomenclature names, the associated set of desired properties, the extracted ingredient information, and the determined functional ingredient category;
determining, by the trained ML model, a synergistic effect of the individual ingredient and at least one additional, potential ingredient in the requested new formulation;
excluding, from the predicted formulation, the individual ingredient from inclusion in the recommended predicted formulation;
determining a multivariate correlation of the functional ingredient category with the set of desired properties;
determining the synergistic effect based on the determined multivariate correlation of the functional ingredient category with the set of desired properties;
the set of desired properties includes a sunscreen protection factor (SPF) value;
the functional ingredient category is determined based on the set of desired properties;
evaluating at least one of an in vivo or an in vitro SPF;
determining the functional ingredient category that highly correlates with the evaluated at least one of the in vivo or in vitro SFP;
receiving feedback on the generated recommendation of the predicted formulation; and
based on the received feedback, retraining the ML model.

The order of execution or performance of the operations in examples of the disclosure illustrated and described herein is not essential, and may be performed in different sequential manners in various examples. For example, it is contemplated that executing or performing a particular operation before, contemporaneously with, or after another operation is within the scope of aspects of the disclosure. When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

In some examples, a system and computer-implemented method for determining an ingredient functional category is provided.

In some examples, a system and computer-implemented method for recommending relevant product formulation candidates is provided.

What is claimed is:

1. A computer-implemented method, comprising:
receiving a request for a new formulation having a set of desired properties;
determining a functional ingredient category for an individual ingredient in the requested new formulation;
identifying a representative molecular structure of the individual ingredient in the determined functional ingredient category;
generating, by a trained machine learning (ML) model, a predicted set of desired properties of i) the requested new formulation and ii) the identified representative molecular structure of the individual ingredient in the determined functional ingredient category; and
generating, based on the generated predicted set of desired properties, a recommended predicted formulation, the predicted formulation including the identified representative molecular structure corresponding to the set of desired properties of the requested new formulation.

2. The computer-implemented method of claim 1, wherein determining the functional ingredient category further comprises:
determining a set of nomenclature names associated with the set of desired properties, the set of nomenclature names being the individual ingredient;
extracting, from a data source, ingredient information for the individual ingredient; and
based on the extracted ingredient information, determining the functional ingredient category for the requested new formulation.

3. The computer-implemented method of claim 2, further comprising:
generating a data structure comprising the determined set of nomenclature names, the associated set of desired properties, the extracted ingredient information, and the determined functional ingredient category.

4. The computer-implemented method of claim 2, further comprising:
determining, by the trained ML model, a synergistic effect of the individual ingredient and at least one additional, potential ingredient in the requested new formulation; and
excluding, from the predicted formulation, the individual ingredient from inclusion in the recommended predicted formulation.

5. The computer-implemented method of claim 4, wherein determining the synergistic effect further comprises:
determining a multivariate correlation of the functional ingredient category with the set of desired properties; and
determining the synergistic effect based on the determined multivariate correlation of the functional ingredient category with the set of desired properties.

6. The computer-implemented method of claim 1, wherein:
the set of desired properties includes a sunscreen protection factor (SPF) value; and
the functional ingredient category is determined based on the set of desired properties.

7. The computer-implemented method of claim 6, wherein determining the functional ingredient category further comprises:
evaluating at least one of an in vivo or in vitro SPF; and
determining the functional ingredient category that highly correlates with the evaluated at least one of the in vivo or in vitro SFP.

8. The computer-implemented method of claim 1, further comprising:
receiving feedback on the generated recommendation of the predicted formulation; and
based on the received feedback, retraining the ML model.

9. A system, comprising:
a memory;
a transceiver configured to receive a request for a new formulation having a set of desired properties; and
a processor coupled to the memory and configured to:
determine a functional ingredient category for an individual ingredient in the requested new formulation;
identify a representative molecular structure of the individual ingredient in the determined functional ingredient category;
generate, by a trained machine learning (ML), a predicted set of desired properties of i) the requested new formulation and ii) the identified representative molecular structure of the individual ingredient in the determined functional ingredient category; and
generate, based on the generated predicted set of desired properties, a recommended predicted formulation, the recommended predicted formulation including the identified representative molecular structure corresponding to the set of desired properties of the requested new formulation.

10. The system of claim 9, wherein, the determine the functional ingredient category, the processor is further configured to:
determine a set of nomenclature names associated with the set of desired properties, the set of nomenclature names being the individual ingredient;
extract, from a data source, ingredient information for the individual ingredient; and
based on the extracted ingredient information, determine the functional ingredient category for the requested new formulation.

11. The system of claim 10, wherein the processor is further configured to:
generate a data structure comprising the determined set of nomenclature names, the associated set of desired properties, the extracted ingredient information, and the determined functional ingredient category.

12. The system of claim 10, wherein the processor is further configured to:
determine, by the trained ML model, a synergistic effect of the individual ingredient and at least one additional, potential ingredient in the requested new formulation; and
exclude, from the predicted formulation, the individual ingredient from inclusion in the recommended predicted formulation.

13. The system of claim 12, wherein, to determine the synergistic effect, the processor is further configured to:
determine a multivariate correlation of the functional ingredient category with the set of desired properties; and
determine the synergistic effect based on the determined multivariate correlation of the functional ingredient category with the set of desired properties.

14. The system of claim 9, wherein:
the set of desired properties includes a sunscreen protection factor (SPF) value; and
the functional ingredient category is determined based on the set of desired properties.

15. The system of claim 14, wherein, to determine the functional ingredient category, the processor is further configured to:
evaluate at least one of an in vivo or in vitro SPF; and
determine the functional ingredient category that highly correlates with the evaluated at least one of the in vivo or in vitro SFP.

16. The system of claim 9, wherein the processor is further configured to:
control to receive feedback on the generated recommendation of the predicted formulation; and
based on the received feedback, retrain the ML model.

17. One or more non-transitory computer readable media storing instructions that, when executed by a processor, cause the processor to:
control to receive a request for a new sunscreen protection factor (SPF) formulation having a set of desired properties;
determine a functional ingredient category for an individual ingredient in the requested new formulation, wherein the functional ingredient category includes at least one of an SPF value, a UVA filter, or a UVB filter;
identify a representative molecular structure of the individual ingredient in the determined functional ingredient category;
generate, by a trained machine learning (ML), a predicted set of desired properties of i) the requested new SPF formulation and ii) the identified representative molecular structure of the individual ingredient in the determined functional ingredient category; and
generate, based on the generated predicted set of desired properties, a recommended predicted SPF formulation, the recommended predicted SPF formulation including the identified representative molecular structure corresponding to the set of desired properties of the requested new SPF formulation.

18. The one or more non-transitory computer readable media of claim 17, further storing instructions that, when executed by the processor, further cause the processor to:
determine a set of nomenclature names associated with the set of desired properties, the set of nomenclature names being the representative ingredients; and
extract, from a data source, ingredient information for the representative ingredients.

19. The one or more non-transitory computer readable media of claim 18, further storing instructions that, when executed by the processor, further cause the processor to:
determine, by the trained ML model, a synergistic effect of the representative ingredients and additional, potential ingredients in the requested new SPF formulation; and
exclude, from the recommended predicted SPF formulation, one of the representative ingredients from inclusion in the recommended predicted SPF formulation.

20. The one or more non-transitory computer readable media of claim 18, further storing instructions that, when executed by the processor, further cause the processor to:
evaluate at least one of an in vivo or in vitro SPF; and
based on the extracted ingredient information, determine the functional ingredient category for the requested new SPF formulation highly correlates with the evaluated at least one of the in vivo or in vitro SFP.
